(19) United States
(12) Patent Application Publication
(10) Pub. No.: US 2002/0160526 A1
(43) Pub. Date: Oct. 31, 2002

(54) PROCESS FOR ISOLATING A TARGET BIOLOGICAL MATERIAL, CAPTURE PHASE, DETECTION PHASE AND REAGENT

(76) Inventors: ABDELHAMID ELAISSARI, LYON (FR); DAVID DURACHER, SAINT NOM LA BRETECHE (FR); CHRISTIAN PICHOT, CORBAS (FR); FRANCOIS MALLET, VILLEURBANNE (FR); ARMELLE NOVELLI-ROUSSEAU, SEYSSINS (FR)

Correspondence Address: OLIFF & BERRIDGE, PO BOX 19928, ALEXANDRIA, VA, 22320

(21) Appl. No.: 09/403,085

(22) PCT Filed: Apr. 16, 1998
(86) PCT No.: PCT/FR98/00772

(30) Foreign Application Priority Data
Apr. 16, 1997 (FR) 97/04923

(51) Int. Cl. G01N 33/543

(52) U.S. Cl. 436/518

(57) ABSTRACT

The invention concerns a method for isolating a target biological material contained in a sample, consisting in the following steps: providing a capture phase, in microparticulate or linear form, consisting of at least a first particulate or linear polymer, with apparent hydrophile character and first complexing groups, the latter being bound by co-ordination to a first transition metal, which is itself bound to a frist biological entity capable of specifically recongnising the target biological material; contacting said target biological material with at least the capture phase; and detecting the capture phase-target biological material complex, optionally with a detection phase, in microparticulate or linear form, and consisting of at least a second particulate or linear polymer, with apparent hydrophile character and second complexing groups, the latter being bound by co-ordination to a second transition metal, which is itself bound to a second biological entity capable of specifically recognizing the target biological material, and a marker.

The present invention relates to the isolation or detection of a biological material, referred to as the target biological material, contained in a sample, by means of a process using a capture phase, and optionally a detection phase, according to which said material is exposed to the capture phase at least, and the capture phase/target biological material complex formed is then detected, optionally with said detection phase.

In the presentation of the invention which follows, reference is made in particular to the isolation of a target protein biological material, but, needless to say, the scope of the invention should not be limited thereto.

Thus, according to the invention, the expression “biological material” means, in particular, a protein or glycoprotein material such as an antigen, a hapten, an antibody, a protein, a peptide, an enzyme or a substrate, and fragments thereof; but also a nucleic material such as a nucleic acid (DNA or RNA), a nucleic acid fragment, a probe or a primer; a hormone.

In accordance with the article by M. Kempe et al. (1), a process is known for capturing a target protein which contains polyhistidine sequences, namely RNase A, according to which the high affinity of the imidazole group of histidine for metals is used.

This process comprises the following steps:

a capture phase is used consisting of silica particles functionalized with methacrylate groups,

a target protein and a metal-complexing agent, namely N-(4-vinyl)benzyliminodiacetic acid (VBIDA), are placed in contact with a metal, in order to obtain a complex resulting from coordination bonding between the metal and the imidazole groups of the histidine, and coordination bonding between the metal and the carboxyl groups of VBIDA, and

said functionalized silica particles are placed in contact with the complex formed above.

This immobilization process does not lead to optimum binding of the target protein.

Document U.S. Pat. No. 4,246,350 describes a process for immobilizing an enzyme using a capture phase which consists of a macroporous polymer containing complexing groups linked to a transition metal. The drawback of such a capture phase results directly from the macroporous nature of the polymer. The reason for this is that, although this macroporous nature makes it possible to maximize the adsorption of the enzyme onto the capture phase, it becomes disadvantageous at the time of isolation of the enzyme using a detection phase, since the proportion of enzyme adsorbed in the polymer pores will not be accessible to said detection phase.

According to the present invention, a process is provided for isolating a target biological material, using a capture phase such that it makes it possible to optimize the binding of this material on this phase, while at the same time reducing, or even eliminating, any side reaction of adsorption of said material onto said capture phase. The interaction between the capture phase is specific, thus making it possible, during isolation, to detect the proportion of biological material effectively bound to the capture phase.

For this purpose, the process for isolating a target biological material uses a capture phase which has the following properties:

it is in microparticulate form or in linear form,

it consists of at least one first particulate or linear polymer, of hydrophilic apparent nature, and first complexing groups, linked covalently,

the first complexing groups are linked by coordination to a first transition metal,

the first transition metal is itself linked by chelation to a first biological species which is capable of specifically recognizing the target biological material.

According to one variant of the process of the invention, the capture phase defined above comprises a marker, in order to obtain a detection phase.

According to another variant of the process, a detection phase is also used which has the following properties:

it is in microparticulate or linear form,

it consists of at least one second particulate or linear polymer, of hydrophilic apparent nature, and second complexing groups,

the second complexing groups are linked by coordination to a second transition metal,

the second transition metal is itself linked by chelation to a second biological species capable of specifically recognizing the target biological material, and a marker,

it comprises a marker.

According to the invention, the term “microparticulate” means in the form of particles not more than 10 μm in size. Preferably, they do not exceed 5 μm in size.

The first and/or second particulate or linear polymer is advantageously a hydrophilic polymer, and in particular a functionalized polymer obtained by polymerization of a water-soluble monomer, of acrylamide, of an acrylamide derivative, of methacrylamide or of a methacrylamide derivative, of at least one crosslinking agent and of at least one functional monomer.

In order to obtain this advantageous polymer, the water-soluble monomer is preferably chosen from N-isopropylacrylamide, N-ethylmethacrylamide, N-n-propylacrylamide, N-n-propylmethacrylamide, N-n-iso-propylmethacrylamide, N-cyclopropylacrylamide, N,N-diethylacrylamide, N-methyl-N-isopropylacrylamide and N-methyl-N-n-propylacrylamide, the monomer preferably being N-isopropylacrylamide (NIPAM). The functional monomer(s) preferably belong(s) to the group corresponding to formula (I) below:

CH₂=C(Z)−(X)ₘ−(P)ₚ−(Y)ₙ(I)

in which:

Z represents H, a C1—C5 alkyl radical or a benzyl, —COOH or —CO—NH—CH(CH₃)₂ radical,

Y represents —CH₂—COOH, —N(CH₂—COOH)₂,

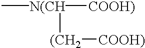

(CH₂COOH), or —(CH₂—CH₂—NH₂)₂,

x represents —NH(CH₂—CH₂—), —N(CH₂—CH₂—)2, —N(CH₂—COOH) (CH₂—CH₂—), or CH(COOH)—,

R represents a linear hydrocarbon-based chain, optionally interrupted with at least one hetero atom such as 0 or N,

m and p are each an integer which, independently of each other, are equal to 0 or 1, and

n is an integer ranging between 1 and 3.

By way of example, the functional monomer is chosen from carboxylic acids, optionally containing nitrogen, itaconic acid, acrylic derivatives and methacrylic derivatives.

As stated previously, the capture phase of the invention can be in microparticulate form or in linear form.

When it is particulate, it can only consist of said particulate polymer, or alternatively it can contain a particulate support such as an organic or inorganic, hydrophilic or hydrophobic core, coated with said first polymer in particulate and/or linear form.

Said core is advantageously chosen from the group comprising polystyrene, silica and metal oxides. It can also comprise a magnetic compound.

The capture phase can also comprise a flat support, partially or totally coated with the first polymer in particulate and/or linear form.

As the examples of the present description will illustrate, the first and [lacuna] second preferred particulate polymer of the invention is poly(N-isopropylacrylamide) (PNIPAM) comprising complexing groups derived from itaconic acid or from maleic acid-co-methyl vinyl ether.

The first and/or second transition metal is advantageously chosen from zinc, nickel, copper, cobalt, iron, magnesium, manganese, lead, palladium, platinum and gold.

According to a preferred embodiment of the process of the invention, the placing in contact of the first biological species with the capture phase and/or the placing in contact of the second biological species with the detection phase, is carried out at a pH above or equal to the isoelectric point of said first and second biological species, respectively.

The expression “biological species” means a biological material as defined above, in isolated form, and presenting, with the target biological material, an affinity to form with said material a complex of the antigen-antibody, enzyme-substrate, hormone-receptor, DNA-DNA, RNA-RNA, etc. type.

Advantageously, the first biological species is a protein. By way of example, it is the protein p24 or gp160 of HIV, for the purpose of isolating, from the serum of a patient, antibodies directed against one or other of these proteins.

The first and/or the second biological species comprises a portion capable of reacting with a transition metal, this portion preferably consisting of a histidine-rich and/or cysteine-rich region.

The sites of affinity of the biological species for the transition metal ions advantageously consist of sites rich in amino acids chosen from histidine, cysteine, tyrosine, tryptophan and phenylalinine.

The sites can be in the form of sequences of said identical or different, contiguous or non-contiguous, but neighboring amino acids.

These sites can exist naturally in the biological species, in particular when it is a protein. Alternatively, they can be “reported” beforehand into the biological species, in the form of “tag”, a definition of which is given below, according to techniques which are well known to those skilled in the art, such as the technique used for the purification of proteins by the IMAC (Immobilized Metal Ion-Affinity Chromatography) process on resins (2, 3). By way of example, such sites can be incorporated into a proteinic biological species and in particular a protein, by genetic engineering, in order to obtain recombinant proteins.

A “tag” can be defined as a reported sequence of amino acids, i.e. a sequence added to the original biological species, which is introduced at a preferred site of the original sequence, where it is exposed in a pertinent manner with respect to its chelation with the transition metal. This sequence contains amino acids chosen from those mentioned above, which are distributed inside the sequence, either contiguously (in particular two abovementioned contiguous amino acids, preferably 6 abovementioned contiguous amino acids), or with a sufficient density (in particular 25%, preferably greater than or equal to 33%). A “tag” which consists of a series of 6 contiguous histidine and/or cysteine residues will be preferred.

According to the process of the invention, a target biological material can be isolated by means of an agglutination reaction using a capture phase described above.

The marker for the detection phase is advantageously chosen from the group consisting of an enzyme, biotin, iminobiotin, a fluorescent component, a radioactive component, a chemiluminescent component, an electron-density component, a magnetic component, an antigen, a hapten and an antibody.

According to the process of the invention, a target biological material can be isolated by means of the ELISA technique using a capture phase and a detection phase, which are described above.

The invention also relates to:

a phase for capturing a target biological material, in microparticulate or linear form and consisting of at least one first particulate or linear polymer, with hydrophilic apparent nature and first complexing groups, the latter being linked by coordination to a first transition metal, which is itself linked to a first biological species capable of recognizing the target biological material,

a phase for detecting a target biological material, in microparticulate or linear form and consisting of at least one second particulate or linear polymer, with hydrophilic apparent nature and second complexing groups, these groups being linked by coordination to a second transition metal, which is itself linked to a second biological species capable of recognizing the target biological material, and a marker,

a reagent for isolating a target biological material, comprising a capture phase and optionally a detection phase as defined above,

each of the capture phase and detection phase having the properties defined above.

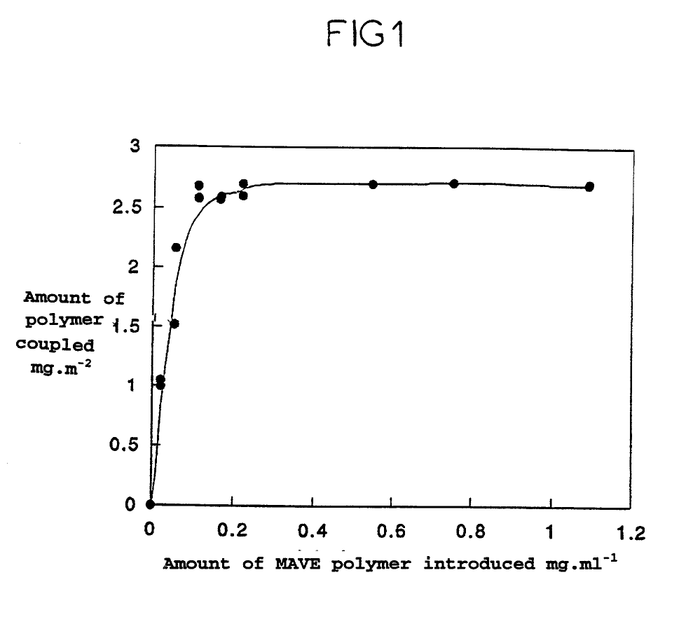

The characteristics and advantages of the present invention are illustrated below by Examples 1 to 5 and FIGS. 1 to 3 according to which:

FIG. 1 represents an isotherm for the coupling of the MAVE polymer with particulate polymer poly-(St-NIPAM-AEM) particles.

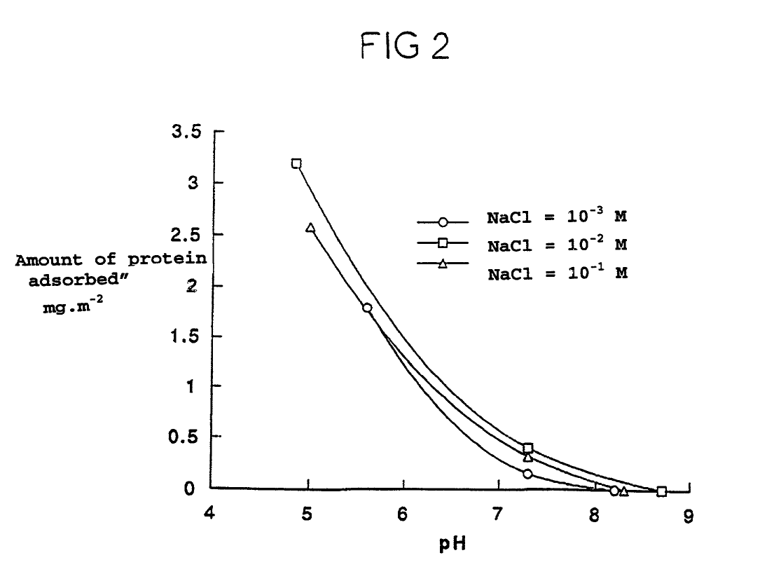

FIG. 2 represents the variation in the amount of protein RH24 adsorbed onto a particulate polymer poly-(St-NIPAM-MAVE) as a function of the pH and of the salinity of the medium.

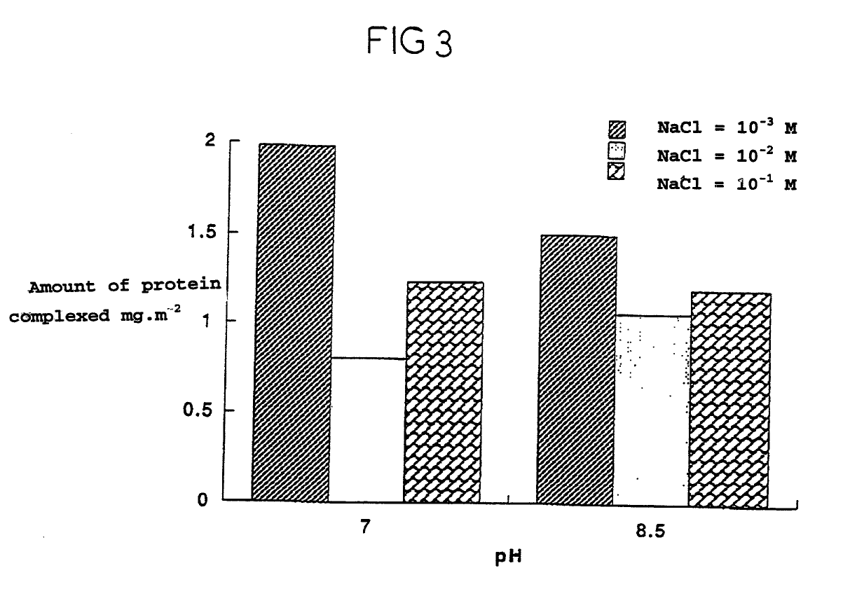

FIG. 3 represents the amount of protein RH24 complexed with a particulate polymer poly-(St-NIPAM-MAVE) as a function of the pH and of the salinity of the medium and for a Zn²⁺ ion concentration of the order of 0.3 M.

### EXAMPLE 1

### Reagents used for the preparation of the capture phase of the invention

Monomer:

99% styrene (Janssen Chemica, refl3 279-87), Mw=104.5 g.mol⁻¹

It is used after purification by distillation under vacuum.

N-isopropylacrylamide (NIPAM) (Kodak ref. 10 982), Mw=113.16 g.mol⁻¹

It is recrystallized before use, as follows. It is dissolved in a hexane/toluene mixture (60/40, v/v).

Functional monomer:

2-aminoethylmethacrylate (AEM) chloride (Kodak ref. 18513), Mw=165.62 g.mol⁻¹

It is used without recrystallization.

Crosslinking agent:

N,N-methylenebisacrylamide (MBA) (Amilabo ref. 10897), Mw=271.19 g.mol⁻¹

It is used without recrystallization.

Primer:

2,2′-azobis(2-amidinopropane) hydrochloride (V50) (Wako trade name), Mw=271.19 g.mol⁻¹

V50 is recrystallized before use, as follows. The primer is dissolved in a 60/40 mixture of water and acetone. The solution is filtered under vacuum with a yield of 30%.

Potassium persulfate (Prolabo), Mw=270.32 g.mol⁻¹

It is used without recrystallization.

Complexing groups:

itaconic acid (Aldrich), Mw=132 g.mol⁻¹

It is used without recrystallization.

Maleic anhydride-co-methyl vinyl ether (MAVE)

(Polysciences)

It is used without recrystallization.

### EXAMPLE 2

### Synthesis of the functionalized polymer poly(N-isopropylacrylamide)-itaconic acid

4.38 g of N-isopropylacrylamide, 200 g of water, 0.37 g of MBA, 0.5 g of itaconic acid and 0.45 g of acrylamide are placed in a 250 ml thermostatically controlled reactor. The mixture is kept stirring at 300 revolutions per minute under an atmosphere of nitrogen and at a temperature of 70° C. Potassium persulfate (0.05 g), a water-soluble primer, is introduced (dissolved in 5 g of water) into the solution at the last moment in order to start the polymerization reaction.

The polymerization reaction is continued for 5 hours under the same conditions.

The degree of conversion of the polymerization is evaluated to 98%.

The functionalized polymer obtained has the following features:

the particle diameter, measured by dynamic light scattering, is 1500 nm,

the assay of the surface functions, followed by conductimetry, gave 0.3 mmol/g of latex of weak acid groups (—COOH).

### EXAMPLE 3

### Modification of the aminohydrophilic particles by grafting the complexing linear polymer poly-MAVE

1) Synthesis of the particulate polymer poly(styrene-NIPAM)

a) Preparation of the hydrophilic particulate polymer

According to this example, the preparation consists in:

in a first stage, combining a polymer poly(St-NIPAM) containing the base monomers, i.e. styrene and NIPAM, according to a polymerization in a closed reactor, with 200 g of water, 18 g of styrene, 2 g of NIPAM and 0.2 g of V50, followed by

in a second stage, adding, to a given degree of conversion, the functional monomer (AEM), alone or in the presence of the base reagents, i.e. 5 g of NIPAM, 0 to 4% of AEM (relative to the NIPAM), 0.122 g of VSO and 0.069 g of BA.

This technique makes it possible to optimize the surface incorporation of a functional monomer. The synthesis conditions are the same as those for the polymerization in a closed reactor, i.e. constant temperature and stirring.

b) Properties of the particulate polymer obtained

The results regarding the structure of the polymer obtained, its size and its polydispersity are collated in Table 1 below.

TABLE 1

| Name of | | (nm) | (nm) | Hair | (nm) | |
|---|---|---|---|---|---|---|
| the | AEM | 20° C. | 50° C. | (nm) | MET | Ip |
| polymer | % | (a) | (a) | (b) | (c) | (c) |
| DD10 | 0 | 603 | 364 | 119 | 288 | 1.012 |
| DD15 | 1 | 421 | 327 | 47 | 333 | 1.008 |
| DD12 | 2 | 484 | 334 | 75 | 302 | 1.004 |
| DD11 | 3 | 358 | 315 | 21 | 303 | 1.005 |

(a): Diameter determined by dynamic light scattering at 20 ° C. and at 50 ° C.
(b): The hair corresponds to the thickness of PNIPAM at the surface of the particles
(c): Diameter and polydispersity index obtained by electron microscopy.

The degree of functionalization of the polymers obtained, expressed by the results of the assay of the amine functions present at the surface of the polymers, are given in Table 2 below.

TABLE 2

| | AEM (%) | SPDP* |
|---|---|---|
| Name of the polymer | introduced | mmol.m⁻² |
| DD10 | 0 | 0.75 |
| DD15 | 1 | 1.44 |
| DD12 | 3 | 2.99 |
| DD11 | 4 | 2.76 |

*: Charge density calculated using the size at 20° C. determined by dynamic light scattering.

2) Grafting of poly-MAVE to the aminated particles

Complexing groups are bound covalently to the polymers obtained according to 1), these complexing groups consisting, according to the present example, of groups derived from MAVE (Maleic Anhydride-co-Methyl Vinyl Ether), which is a linear polymer.

The use of MAVE has two advantages: on the one hand, it allows, by virtue of its highly reactive anhydride functions, easy coupling with the amines present at the surface of the particulate polymer, and, on the other hand, once the coupling has been achieved, it exposes several complexing dicarboxylic functions, which will interact with a transition metal (Zn, Ni, Cu, Co, etc.).

MAVE is used as a solution in anhydrous DMSO in order to avoid hydrolysis of the anhydride functions via which the coupling reaction with the amine functions of the particulate polymers is possible. The coupling reaction should be carried out in a basic medium in order to avoid protonation of the amine functions of the polymers. The buffer used is a borate buffer of pH 8.2 and with an ionic strength of 10⁻² M. The coupling medium should not exceed 10% by volume of DMSO.

The results, which are given in FIG. 1, show a good correlation between the two analysis methods. The initial slope of the coupling isotherm shows that the reaction is complete for small amounts of MAVE introduced. The value of the plateau is 2.75 mg.m⁻² and is reached very quickly for low concentrations of MAVE.

### EXAMPLE 4

### Complexation of a transition metal with the polymer containing complexing groups

The introduction of a transition metal into a solution of the polymer containing complexing groups, obtained according to Example 2 or 3, should allow the binding of the metal by complexation to the particles. This complexation takes place by means of the oxygen atoms of the anhydride functions. The presence of lone pairs on the oxygen atoms makes it possible to form coordination bonds with the transition metal.

The metal used (Zn²⁺) is introduced into a solution of the polymer in order to obtain a concentration of metal ion solution of 10⁻⁴ M. The excess metal cation which is in solution is removed by successive centrifugations.

### EXAMPLE 5

### Complexation of the protein RH24 used as biological species to obtain a capture phase of the invention

The biological species selected for this example is the recombinant protein (referred to as RH24) modified at the N-terminal with a histidine “tag” (sequence of six contiguous histidine residues) (5). This protein has a mass of 27.103 g.mol⁻¹ and an isoelectric point of 6.1. This modification was exploited to achieve the complexation of the protein on a particulate support, in order to obtain a capture phase of the invention.

In order to be able to determine the concentration of protein complexed on the latex, studies of adsorption of the protein were carried out in parallel.

As the state of the art shows, these are electrostatic interactions which govern the adsorption of the proteins onto a hydrophyilic polymer (6). Thus, the effect of the ionic strength and of the pH on the amount of proteins adsorbed was studied in order to determine the conditions for which the adsorption is negligible, or even nonexistent.

FIG. 2 shows the adsorption of the protein RH24 onto poly(St-NIPAM-MAVE) obtained according to Example 3.

According to FIG. 2, it is seen that the degree of adsorption of RH24 is highly pH-dependent.

A similar study was carried out for the complexation by varying the same parameters. FIG. 3 shows the results of the complexation depending on the pH, for various ionic strengths and for constant concentrations of complexing ion (Zn²⁺).

As seen in this figure, complexation of the protein with poly(St-NIPAM-MAVE) in the presence of zinc is little dependent on the pH, except for the low ionic strengths.

These results make it possible to determine optimum conditions for complexation at the expense of adsorption. Thus, a pH above or equal to 7 makes it possible to have virtually no adsorption while at the same time having a complexation of close to 1.5 mg.m⁻². As regards the ionic strength, this has to be minimal in order to promote the complexation.

### BIBLIOGRAPHY

(1) Kempe M., Glad M. & Mosbach K., Journal of molecular recognition, 8, 35 (1995)

(2) Porath J., Carlsson., Olsson., Belfrage J., Nature, 258, 598 (1975)

(3) Porath J., Trends Anal. Chem., 7, 254 (1988)

(4) Hiroshi Inomata et al., Macromolecules, 27, 6459-6464 (1994)

(5) Cheynet V., Verrier B., Mallet F., proteine expression and purification, 4, 367 (1993)

(6) Suzawa T., Shirahama H., Advances in Colloid and Interface Science, 35, 139 (1991).

1. Process for isolating a target biological material contained in a sample, according to which a capture phase is used, said target biological material being placed in contact with at least the capture phase, and the capture phase/target biological material complex is detected,
said process being characterized in that,
the capture phase is in microparticulate or linear form and consists of at least one first particulate or linear polymer, with a hydrophilic apparent nature and first complexing groups, these groups being linked by coordination to a first transition metal, which is itself linked to a first biological species capable of specifically recognizing the target biological material.

2. Process according to claim 1, characterized in that the capture phase comprises a marker in order to obtain a detection phase.

3. Process according to claim 1, characterized in that a detection phase is also used, which is in microparticulate or linear form and consists of at least one second particulate or linear polymer, of hydrophilic apparent nature, and second complexing groups, these groups being linked by coordination to a second transition metal, which is itself linked to a second biological species capable of specifically recognizing the target biological material, and a marker.

4. Process according to claim 1 or 3, characterized in that the first and/or the second polymer is chosen from the group of hydrophilic polymers.

5. Process according to claim 4, characterized in that the first and/or the second polymer is a functionalized polymer obtained by polymerization of a water-soluble monomer, of acrylamide, of an acrylamide derivative, of methacrylamide or of a methacrylamide derivative, of at least one crosslinking agent and of at least one functional monomer.

6. Process according to claim 5, characterized in that the water-soluble monomer is chosen from N-isopropylacrylamide, N-ethylmethacrylamide, N-n-propyl-acrylamide, N-n-propylmethacrylamide, N-n-isopropyl-methacrylamide, N-cyclopropylacrylamide, N,N-diethylacryl-amide, N-methyl-N-isopropylacrylamide and N-methyl-N-n-propylacrylamide, the first monomer preferably being N-isopropylacrylamide (NIPAM).

7. Process according to claim 5, characterized in that the functional monomer corresponds to formula I below:
CH₂=C(Z)−(X)ₘ−(R)ₚ−(Y)ₙ (I)
in which
Z represents H, a C1-C5 alkyl radical or a benzyl, —COOH or —CO—NH—CH(CH,)₂ radical,
Y represents —CH₂—COOH, —N(CH₂—COOH)₂, 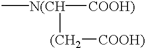
(CH₂—COOH), or —N(CH₂—CH₂—NH₂)₂,
x represents —NH(CH₂—CH₂—), —N(CH₂—CH₂—)₂, —N(CH₂—COOH) (CH₂—CH₂—), or CH(COOH)—,
R represents a linear hydrocarbon-based chain, optionally interrupted with at least one hetero atom such as O or N,
m and p are each an integer which, independently of each other, are equal to 0 or 1, and
n is an integer ranging between 1 and 3.

8. Process according to claim 7, characterized in that the functional monomer is chosen from carboxylic derivatives, optionally containing nitrogen, itaconic acid, acrylic derivatives and methacrylic derivatives.

9. Process according to any one of claims 1 to 8, characterized in that the capture phase and/or the detection phase is in microparticulate form and in that the average particle size is not more than 5 μm.

10. Process according to claim 1, characterized in that the capture phase also comprises a flat or particulate support.

11. Process according to claim 10, characterized in that the support is particulate and consists of an organic or inorganic, hydrophilic or hydrophobic core.

12. Process according to claim 11, characterized in that said core is chosen from the group comprising polystyrene, silica and metal oxides.

13. Process according to claim 11 or 12, characterized in that said core also contains a magnetic compound.

14. Process according to any one of claims 11 to 13, characterized in that said core is coated with said first polymer, this polymer being linear.

15. Process according to any one of claims 11 to 13, characterized in that said core is coated with said polymer, said polymer being particulate.

16. Process according to claim 1 or 3, characterized in that the first and/or the second polymer is poly(N-isopropylacrylamide) and the complexing groups are derived from itaconic acid or from maleic anhydride-co-methyl vinyl ether.

17. Process according to claim 1 or 3, characterized in that the first and/or second transition metal is chosen from zinc, nickel, copper, cobalt, iron, magnesium, manganese, lead, palladium, platinum and gold.

18. Process according to claim 1 or 3, characterized in that the placing in contact of the first biological species with the capture phase and/or the placing in contact of the second biological species with the detection phase is carried out at a pH above or equal to the isoelectric point of said first and second biological species, respectively.

19. Process according to claim 1 or 3, characterized in that the first and/or the second biological species is rich in histidine and/or cysteine.

20. Process according to claim 1 and any one of claims 4 to 19, characterized in that an agglutination reaction is used.

21. Process according to claim 2 or 3, characterized in that the marker for the detection phase is chosen from the group consisting of an enzyme, biotin, iminobiotin, a fluorescent component, a radioactive component, a chemiluminescent component, an electron-density component, a magnetic component, an antigen, a hapten and an antibody.

22. Process according to claim 2 or 3 and any one of claims 4 to 19 or 21, characterized in that the ELISA technique is used.

23. Phase for capturing a target biological material, characterized in that it is in microparticulate or linear form and consists of at least one first particulate or linear polymer, of hydrophilic apparent nature, and first complexing groups, these groups being linked by coordination to a first transition metal, which is itself linked to a first biological species capable of recognizing the target biological material.

24. Phase for detecting a target biological material, characterized in that it is in microparticulate or linear form and consists of at least one second particulate or linear polymer, of hydrophilic apparent nature, and second complexing groups, these groups being linked by coordination to a second transition metal, which is itself linked to a second biological species capable of recognizing the target biological material, and a marker.

25. Reagent for isolating a target biological material, comprising a capture phase according to claim 23 and/or a detection phase according to claim 24.
